(19) United States
(12) Patent Application Publication
(10) Pub. No.: US 2023/0053336 A1
(43) Pub. Date: Feb. 23, 2023

(54) NUCLEATION METHOD OF PRODUCING POLYCAPROLACTONE POWDER

(71) Applicant: JABIL INC., St. Petersburg, FL (US)

(72) Inventors: Thomas George Gardner, St. Petersburg, FL (US); Victoria Hannah Pyle, St. Petersburg, FL (US); Travis Lee Hislop, St. Petersburg, FL (US)

(21) Appl. No.: 17/820,405

(22) Filed: Aug. 17, 2022

Related U.S. Application Data
(60) Provisional application No. 63/234,812, filed on Aug. 19, 2021.
(60) Provisional application No. 63/265,641, filed on Dec. 17, 2021.

(51) Int. Cl. B29C 64/153; B33Y 10/00; B33Y 70/00; C08J 3/14

(52) U.S. Cl. CPC B29C 64/153; B33Y 10/00; B33Y 70/00; C08J 3/14; B29K 2067/046

(57) ABSTRACT

Disclosed is a method of preparing a polycaprolactone powder possessing properties making it well-suited to powder bed fusion 3D printing processes. The polycaprolactone powder disclosed herein has an enthalpy of fusion between 80 J/g and 140 J/g. The polycaprolactone powder described herein has a D90 between 20 microns and 150 microns. The polycaprolactone powder described herein contains a detectable amount of a biocompatible solvent, a bioresorbable solvent, and/or ethyl lactate.

### CROSS REFERENCE TO RELATED PATENT APPLICATIONS

This patent application claims priority to U.S. Provisional Patent Application No.

63/234,812, filed on Aug. 19, 2021, and U.S. Provisional Patent Application No. 63/265,641, filed on Dec. 17, 2021, which are incorporated by reference herein in their entirety.

### FIELD

This disclosure relates to the production of a polycaprolactone (also referred to as PCL) powder. The disclosed polycaprolactone powder may be used as a build material for producing three-dimensional objects via 3D printing or other known manufacturing methods, such as molding. The disclosed polycaprolactone powder may be suitable for producing implantable objects via selective laser sintering (SLS).

### BACKGROUND

Biocompatible and bioresorbable polymers may be used to make medical implants that are non-toxic to the human body.

3D printers create solid, three-dimensional objects by joining adjacent materials together, for example by melting and/or sintering adjacent materials so that they solidify together upon cooling. 3D printers typically follow the instructions of a computer-aided design (CAD) model and build objects layer by layer. 3D printing is a type of additive manufacturing. Additive manufacturing may include material extrusion, powder bed fusion, binder jetting, vat photopolymerization, sheet lamination, directed energy deposition, and material jetting.

Selective laser sintering (SLS) is one type of 3D printing that may be used to create medical implants, among other things. SLS machines may require a print/build material to be in the form of a powder with a specific particle size distribution and other characteristics. The machines may also require the print material to have a certain amount of flowability. Flowability may allow a print material to evenly spread with each new layer of build material that is laid down before applying electromagnetic energy (typically in the form of laser energy) to sinter predefined regions.

3D print applications may include: SLS (selective laser sintering), MJF (multi jet fusion), HSS (high speed sintering), and electrophotography.

Flow aids may be added to improve an SLS print material's flowability. However, it may be undesirable to add certain flow aids to medical implants because their addition might result in adverse effects in a patient's body. Therefore, when producing an SLS powder for making medical implants, in some cases it may be desirable to have good particle sphericity to minimize or eliminate the need for a flow aid.

This disclosure relates to a solvent precipitation method of producing partially crystalline polycaprolactone powder that may be suitable for use in an SLS machine.

### SUMMARY

A number of variations within the scope of the claims may include processes, compositions, and articles of manufacture that relate to the preparation of a PCL powder and its use thereof in additive manufacturing processes, including PBF processes.

At least one variation may include a powder comprising polycaprolactone particles. The powder having greater than 90 volume percent of the particles with a particle size between 20 microns and 150 microns. The powder having a detectable amount of solvent and a detectable amount of a nucleator, where the solvent is a biocompatible solvent or a bioresorbable solvent In some variations the solvent is ethyl lactate. In some variations the nucleator is hydroxyappetite. In some variations, greater than 90 volume percent of the polycaprolactone particles have a sphericity greater than 0.75. In another variation, greater than 90 volume percent of the polycaprolactone particles have a sphericity greater than 0.80. In some variations, the volume percent of polycaprolactone particles having a particle size less than 20 microns is zero or undetectable. In some variations, the powder has a peak melting temperature of about 55° C. to about 65° C. and an enthalpy of fusion of about 90 J/g to about 120 J/g. In some variations, the powder has a recrystallization peak of about 15° C. to about 35° C. In some variations, the powder has a degradation temperature of about 250° C. to about 425° C. In some variations greater than 96 number percent of the polycaprolactone particles have a particle size that is less than 125 microns. In some variations, the polycaprolactone particles have a moisture content that is adjusted to and maintained between 0.5% w/w and 5% w/w.

At least one variation may include a method of preparing PCL powder that may include combining polycaprolactone in a polar organic solvent, dissolving the polycaprolactone in the polar organic solvent forming a solution, cooling the solution to a temperature that causes at least a portion of the dissolved polycaprolactone to precipitate. A nucleator may be added to the solution to promote precipitation. The powder is separated from the solution, leaving behind a second, more dilute PCL solution, as well as contaminants from the raw PCL; for example, residual catalyst, initiator, polymerization solvent, monomer, and oligomers. The separated powder may then be washed and dried. In some variations, the method further includes heating the combined polycaprolactone and the polar organic solvent. In some variations, the method further includes a separation step that separates dry polycaprolactone particles having a particle size less than 150 microns from larger dry polycaprolactone particles to form a sized polycaprolactone. In some variations, the percent of nucleator in the combined polycaprolactone/nucleator mixture is between about 0.5 mass percent and 10 mass percent. In some variations, the nucleator is hydroxyappetite. In some variations, polar organic solvent is selected from the group consisting of: ethyl acetate, ethyl lactate, γ-valerolactone, N,N-dimethylformamide (DMF), N-methyl-2-pyrrolidone (NMP), tetrahydrofuran (THF), dichloromethane (DCM), chloroform; acetone, and dimethyl sulfoxide (DMSO).

At least one variation may include a method of producing a powder comprising polycaprolactone particles including combining polycaprolactone and a polar organic solvent and dissolving the polycaprolactone in the polar organic solvent along with at least one nucleator. The solution may then be cooled to a lower temperature causing at least a portion of the dissolved polycaprolactone to precipitate in the solution. The precipitated polycaprolactone is separated from the solution, washed, and dried. In some variations, the method includes heating the solution.

At least one variation may include a method of additive manufacturing including selectively melting or sintering adjacent polycaprolactone particles. Greater than 95 number percent of the polycaprolactone particles have a particle size less than 125 microns, and greater than 90 volume percent of the polycaprolactone particles have a sphericity greater than 0.75. The polycaprolactone particles contain a detecetable amount of ethyl lactate and a detectable amount of hydroxyappetite. In some variations, the polycaprolactone particles have a moisture content that is adjusted to and maintained between 0.5 and 5% w/w.

At least one variation may include an article that includes polycaprolactone particles. Greater than 90 volume percent of the polycaprolactone particles have a particle size that is between 20 microns and 150 microns. The polycaprolactone particles contain a detectable amount of a nucleator. The polycaprolactone particles contain a detectable amount of a solvent comprising at least one of a biocompatible solvent or a bioresorbable solvent.

At least one variation may include a medical product that includes polycaprolactone particles. Greater than 90 volume percent of the polycaprolactone particles have a particle size that is between 20 microns and 150 microns. The polycaprolactone particles contain a detectable amount of a nucleator. The polycaprolactone particles contain a detectable amount of a solvent comprising at least one of a biocompatible solvent or a bioresorbable solvent.

Powder compositions for use in PBF processes are provided that include PCL powder prepared by such a method. Objects may be prepared by using such PCL powders in a PBF process to form the object.

The disclosed illustrative of variations of apparatuses, systems, and methods provide PCL powder having suitable properties and characteristics for use in SLS, MJF, HSS, and electrophotography 3D-printing applications. An embodiment of the disclosure may provide a precipitated PCL powder formed through precipitating the polymer from a solvent and then employing the precipitated pulverulent polymer in a powder-based 3D-printing process.

Further areas of applicability will become apparent from the description provided herein. The description and specific examples in this summary are intended for purposes of illustration only and are not intended to limit the scope of the disclosure or claims.

Variations may include a powder comprising polycaprolactone particles. In at least one variation, greater than 90 volume percent of the polycaprolactone particles have a particle size that is between 20 microns and 150 microns.

### BRIEF DESCRIPTION OF THE DRAWINGS

FIG. 1 is a flow diagram showing a method of producing polycaprolactone powder, according to at least one variation.

FIG. 2 is a graph which shows results from a thermal gravimetric analysis (TGA) that was performed on a sample of polycaprolactone produced according to at least one variation.

FIG. 3 is a graph which shows a differential scanning calorimetry (DSC) curve of polycaprolactone precipitated according to at least one variation.

FIG. 4 is a graph which shows the particle size volume distribution for an SLS-grade powder that was produced according to at least one variation.

FIG. 5 is a graph which shows the particle size number distribution for an SLS-grade powder that was produced according to at least one variation.

FIG. 6 is a table which shows powder data for a polycaprolactone powder that was produced according to at least one variation.

FIG. 7 is a picture of bars that were SLS printed using polycaprolactone that included 4% w/w (weight/weight) hydroxyapatite (also referred to as HA) according to at least one variation.

FIG. 8A is a graph which shows a tensile plot that was generated by pulling the SLS created polycaprolactone (with 4% w/w hydroxyapatite) tensile bars.

FIG. 8B is a table which shows a summary of the material properties obtained from the tensile testing in FIG. 8A.

FIG. 9 is a graph which shows a DSC curve of the resulting polycaprolactone powder nucleated by hydroxyapatite according to at least one variation.

FIG. 10 is a graph which shows a particle number size distribution according to at least one variation.

FIG. 11 is a graph which shows a particle number size distribution according to at least one variation.

FIG. 12A shows a polycaprolactone powder nucleated with 4% w/w hydroxyapatite according to at least one variation.

FIG. 12B shows a polycaprolactone powder dry blended with 4% w/w hydroxyapatite and allowed to sit for over 24 hours, according to at least one variation.

FIG. 13 is a table which shows a particle size distribution comparison between polycaprolactone precipitated on its own and polycaprolactone precipitated with hydroxyapatite acting as a nucleator, according to at least one variation.

FIG. 14A shows a polycaprolactone puck prepared by a method according to at least one variation.

FIG. 14B shows a polycarprolactone puck prepared by a method according to at least one variation.

FIG. 14C shows a polycarprolactone puck prepared by a method according to at least one variation.

FIG. 14D shows a polycarprolactone puck prepared by a method according to at least one variation.

FIG. 14E shows a polycarprolactone puck prepared by a method according to at least one variation.

FIG. 14F shows a polycarprolactone puck prepared by a method according to at least one variation.

FIG. 14G shows a polycarprolactone puck prepared by a method according to at least one variation.

FIG. 15 is a graph which shows a DSC curve for polycaprolactone powder that was reprecipitated in ethyl lactate, according to at least one variation.

FIG. 16 is a graph which shows a DSC curve for polycaprolactone powder that was reprecipitated in the presence of 4% w/w hydroxyapatite as a nucleator, according to at least one variation.

### DETAILED DESCRIPTION

The following description is merely illustrative in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is illustrative in nature, and thus, the order of the steps may be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

The term ‘or” as used herein, with respect to a list of two or more items, elements, components, or materials, is not indicative of a complete disjunction such that the listed items, elements, components, or materials are mutually exclusive of each other. For example, “X, Y, or Z” does not mean that each of X, Y, Z are mutually exclusive of each other. Two or more of X, Y, Z could partially or completely overlap each other or that at least one of X, Y, or Z could be included in or be a subgenus of at least one of another of X, Y, or Z. As another example, “cells may be grown in monolayer, three dimensions, or on beads” does not mean that cells grown on beads does not include cells grown in three dimensions. As a further example, “at least one of a biocompatible solvent; a bioresorbable solvent; or ethyl lactate” does not mean that ethyl lactate nor a solvent including ethyl lactate is neither a biocompatible solvent nor a bioresorbable solvent; nor does it mean that a biocompatible solvent or a bioresorbable solvent cannot be or include ethyl lactate.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

The particle size of the PCL polymer may affect its use in additive manufacturing processes.

As used herein, D₅₀ (as known as “volume median diameter” or “average particle diameter by volume”) refers to the particle diameter of the powder where 50 vol. % of the particles in the total distribution of the referenced sample have the noted particle diameter or smaller. Similarly, D₁₀ refers to the particle diameter of the powder where 10 vol. % of the particles in the total distribution of the referenced sample have the noted particle diameter or smaller; and D₉₀ refers to the particle diameter of the powder where 90 vol. % of the particles in the total distribution of the referenced sample have the noted particle diameter or smaller. Particle sizes may be measured by any suitable methods known in the art to measure particle size by diameter. The semi-crystalline polymer powder provided herein may have a D₉₀ particle size of less than 150 μm.

As used herein, “layer” is a term of convenience that includes any shape, regular or irregular, having at least a predetermined thickness. In certain embodiments, the size and configuration two dimensions are predetermined, and in certain embodiments, the size and shape of all three-dimensions of the layer are predetermined. The thickness of each layer may vary widely depending on the additive manufacturing method. In certain embodiments the thickness of each layer as formed may differ from a previous or subsequent layer. In certain embodiments, the thickness of each layer may be the same. In certain embodiments the thickness of each layer as formed may be from 0.5 millimeters (mm) to 5 mm.

Certain variations may include forming a plurality of layers in a preset pattern by an additive manufacturing process. In a number of variations, the additive manufacturing may produce two or more layers, or 20 or more layers. The maximum number of layers may vary greatly, determined, for example, by considerations such as the size of the object being manufactured, the technique used, the capacities and capabilities of the equipment used, and the level of detail desired in the final object. For example, 5 to 100,000 layers may be formed, or 20 to 50,000 layers may be formed, or 50 to 50,000 layers may be formed.

The term “powder bed fusing” or “powder bed fusion” is used herein to mean processes wherein the polymer is selectively sintered or melted and fused, layer-by-layer to provide a 3-D object. Sintering may result in objects having a density of less than about 90% of the density of the solid powder composition, whereas melting may provide objects having a density of 90%-100% of the solid powder composition. Use of semi-crystalline polymer as provided herein may facilitate melting such that resulting densities may approach densities achieved by injection molding methods.

Powder bed fusing or powder bed fusion further includes all laser sintering and all selective laser sintering processes as well as other powder bed fusing technologies as defined by ASTM F2792-12a. For example, sintering of the powder composition may be accomplished via application of electromagnetic radiation other than that produced by a laser, with the selectivity of the sintering achieved, for example, through selective application of inhibitors, absorbers, susceptors, or the electromagnetic radiation (e.g., through use of masks or directed laser beams). Any other suitable source of electromagnetic radiation may be used, including, for example, infrared radiation sources, microwave generators, lasers, radiative heaters, lamps, or a combination thereof. In certain embodiments, selective mask sintering (“SMS”) techniques may be used to produce three-dimensional objects. For further discussion of SMS processes, see for example U.S. Pat. No. 6,531,086, the entire contents which are incorporated herein by reference, which describes an SMS machine in which a shielding mask is used to selectively block infrared radiation, resulting in the selective irradiation of a portion of a powder layer. If using an SMS process to produce objects from powder compositions of the present technology, it may be desirable to include one or more materials in the powder composition that enhance the infrared absorption properties of the powder composition. For example, the powder composition may include one or more heat absorbers (e.g., glass fibers or glass microbeads) or dark-colored materials (e.g., carbon black, carbon nanotubes, or carbon fibers).

Also included herein are all three-dimensional objects made by powder bed fusing compositions including the semi-crystalline polymer powder described herein. After a layer-by-layer manufacture of an object, the object may exhibit excellent resolution, durability, and strength. Such objects may include various articles of manufacture that have a wide variety of uses, including uses as prototypes, as end products, as well as molds for end products.

An object may be formed from a preset pattern, which may be determined from a three-dimensional digital representation of the desired object as is known in the art and as described herein. Material may be joined or solidified under computer control, for example, working from a computer-aided design (CAD) model, to create the three-dimensional object.

In particular, powder bed fused (e.g., laser sintered) objects may be produced from compositions including PCL powder using any suitable powder bed fusing processes including laser sintering processes. These objects may include a plurality of overlying and adherent sintered layers that include a polymeric matrix which, in some embodiments, may have reinforcement particles dispersed throughout the polymeric matrix. Laser sintering processes are known, and are based on the selective sintering of polymer particles, where layers of polymer particles are briefly exposed to laser energy and the polymer particles exposed to the laser energy are thus bonded to one another. Successive sintering of layers of polymer particles produces three-dimensional objects. Details concerning the selective laser sintering process are found, by way of example, in the specifications of U.S. Pat. No. 6,136,948 and WO 96/06881, the entire contents of each of which are incorporated herein by reference. However, the semi-crystalline polymer powder described herein may also be used in other rapid prototyping or rapid manufacturing processing of the prior art, in particular in those described above. For example, the semi-crystalline polymer powder may in particular be used for producing moldings from powders via the SLS (selective laser sintering) process, as described in U.S. Pat. No. 6,136,948 or WO 96/06881, via the SIB process (selective inhibition of bonding of powder), as described in WO 01/38061, via 3D printing, as described in EP 0 431 924, or via a microwave process, as described in DE 103 11 438, the entire contents of each of which are incorporated herein by reference.

The fused layers of powder bed fused objects may be of any thickness suitable for selective laser sintered processing. The individual layers may be each, on average, at least 50 μm thick, at least 80 μm thick, or at least 100 μm thick. In a number of variations, the plurality of sintered layers are each, on average, less than 500 μm thick, less than 300 μm thick, or less than 200 μm thick. Thus, the individual layers for some embodiments may be 50 to 500 μm, 80 to 300 μm, or 100 to 200 μm thick. Three-dimensional objects produced from powder compositions of the present technology using a layer-by-layer powder bed fusing processes other than selective laser sintering may have layer thicknesses that are the same or different from those described above.

A number of variations may provide ways to make and use PCL powder having suitable characteristics for use in selective laser sintering (SLS), multi jet fusion (MJF), high speed sintering (HSS), and electrophotographic (EPG) 3D-printing. At least one variation may provide a precipitated PCL powder formed through precipitation of the polymer from a saturated solution of PCL in a polar organic solvent, allowing the polymer to form crystallites, and then employing the precipitated polymer powder in a PBF 3D-printing process. A number of variations of PCL powder may exhibit optimized characteristics for PBF processes, including optimized particle size and dispersity thereof, shape, and crystallinity, while at the same time using a dispersant-free single-solvent process in the manufacture thereof.

Methods of preparing PCL powder may include dissolving bulk PCL in ethyl lactate to form a solution at elevated temperature; cooling the solution to room temperature to form a PCL powder as a precipitate having a D₉₀ value of less than 150 micrometers (microns, or μm); a D₅₀ value of less than or equal to 100 μm, or a D₅₀ value of between 0 to 100 μm. The methods may also yield a product where the particles may exhibit a certain size (about 30 μm to about 40 μm in average diameter), low dispersity, spheroidal shape, and crystalline character suitable for the above- mentioned printing processes in comparison to the results of aforementioned processes. The act of reprecipitation also serves to purify the PCL.

Powder compositions for use in PBF processes are provided that include PCL powder prepared by such a method. Objects may be prepared by using such PCL powders in a PBF process to form the object.

In certain embodiments, a method of preparing PCL powder is provided that includes dissolving bulk PCL in a polar solvent such as an ester; for example, ethyl lactate, to form a first solution of dissolved polymer at a first temperature. The first solution is then cooled to a second temperature, where the second temperature is lower than the first temperature. A portion of the dissolved PCL precipitates as powder from the first solution either en route to, or upon arrival at, the second temperature, leaving behind a second, more dilute PCL solution. The precipitated PCL powder may be separated from a remainder of the second solution, effected for example by gravity filtration, vacuum filtration, or centrifugation. The separated PCL powder may also be washed with water or an organic solvent, provided the wash solvent is miscible with the solvent used for reprecipitation, and that the wash solvent does not dissolve the polymer powder to a deleterious extent (e.g., unacceptably excessive loss of material and/or unacceptably excessive reduction of particle size), and may not a solvent for the polymer powder product at all. The separated PCL powder may also be dried, subsequent to any washing procedure, if applied. In certain embodiments, the polar solvent may include ethyl lactate. In other embodiments, the polar solvent may consist essentially of ethyl lactate. And in still further embodiments, the polar solvent may consist of ethyl lactate.

Various solvent temperatures may be employed in methods of preparing PCL powder by reprecipitation. The dissolving step may include heating PCL in a polar solvent to form the first solution of dissolved PCL at the first temperature, where the first temperature is greater than room temperature. The cooling step may include cooling the first solution to the second temperature, where the second temperature is below the precipitation temperature of the polymer solution, and may be at ambient temperature (“room temperature”) or lower. Ambient (“room”) temperature is understood to be about 20-25° C. (68-77° F.).

Various embodiments of PCL may exhibit the following physical characteristics. The PCL powder may have a D₉₀ particle size of less than about 150 μm. In certain embodiments, the PCL powder may have a D₅₀ of less than about 100 μm. The PCL powder may also have a D₅₀ value from about 1 micrometer to about 100 μm. Particular embodiments include where the PCL powder has a D₅₀ value from about 30 μm to about 40 μm. The PCL powder may be in the form of spheroidal particles.

Melting point and enthalpy of fusion for the polymer powder may be determined using differential scanning calorimetry (DSC); for example, a TA Instruments Discovery Series DSC 250 scanning at 20° C/min.

Percent crystallinity of a polymer may be determined by the ratio of the enthalpy of fusion, as measured by DSC, to the enthalpy of fusion of a theoretical 100% crystalline polymer, which for PCL is reported as having a value of 139.5 J/g (Gupta and Geeta, J. Appl. Polym.. Sci. 2012, 123(4), 1944-1950). Percent crystallinity may also be determined directly by powder x-ray crystallography and correlated to enthalpy of fusion in a directly linear relationship.

Powder flow for the polymer powder may be measured using Method A of ASTM D₁₈₉₅ and was determined using a cone with a 10 mm nozzle diameter.

In some embodiments, the particle size of the polymer powder is determined by laser diffraction as is known in the art. For example, particle size may be determined using a laser diffractometer such as the Microtrac 53500.

In certain embodiments, powder compositions for use in a PBF 3D printing process are provided, where such powder compositions include PCL powder prepared according to the methods provided herein. For example, a powder composition for use in a PBF process may include PCL powder having a D₉₀ particle size of less than about 150 μm, and a D₅₀ value from about 30 μm to about 40 μm. Such powder compositions may include mixtures of PCL powders having different physical characteristics as well as additives and other components as described herein.

In certain embodiments, reprecipitated PCL powder prepared by methods disclosed herein is used in a PBF 3D printing process to form an object. Certain methods of preparing an object include providing PCL powder having a D₉₀ particle size of less than about 150 μm, a D₅₀ value from about 30 μm to about 40 μm. The PCL powder is then used in a PBF process to form the object.

In certain embodiments, one or more objects prepared by an additive manufacturing process are provided. Such methods may include providing PCL powder prepared according to one or more of the methods described herein. The PCL powder is then used in a PBF process to form the one or more objects.

Certain embodiments may include methods for powder bed fusing that use a powder composition including PCL powder to form a three-dimensional object. Due to the good flowability of reprecipitated PCL powder, a smooth and dense powder bed may be formed allowing for optimum precision and density of the sintered object.

In certain embodiments, the method of preparing PCL powder comprises dissolving bulk

PCL in a polar solvent such as ethyl lactate at a temperature above room temperature. Ambient (“room”) temperature is understood to be about 20-25° C. (68-77° F.); as such, the PCL may be dissolved in ethyl lactate above ambient temperature. The PCL is soluble in the ethyl lactate solvent and thus a PCL solution is formed. In general, the solution may be prepared at a temperature above room temperature so that the amount of dissolved PCL is greater than what the solvent is capable of keeping in solution at ambient temperature. Mixing of PCL into ethyl lactate solvent may be carried out in-line or batch. The process may readily be carried out at manufacturing scale. Upon cooling to room temperature (e.g., about 20° C.), the dissolved PCL begins to crystallize and precipitate out of the ethyl lactate solvent resulting in the precipitation of a PCL precipitate.

Following precipitation, the ethyl lactate solvent is removed, for example by filtration or centrifugation. The PCL powder may then be washed with a solvent that is miscible with the reprecipitation solvent and reasonably volatile, for example, water, filtered to remove the wash solvent, and dried with or without application of heat, and with or without application of vacuum. It is further advantageous to use a wash solvent in which PCL is minimally soluble or insoluble.

As provided herein, PCL is dissolved in a polar organic solvent. For example, PCL may be dissolved in the solvent under conditions that result in a saturated solution of PCL, where changing conditions (e.g., lowering the temperature of the solution) result in precipitation of PCL powder therefrom. In certain embodiments, the solvent may include ethyl lactate as well as one or more other esters or one or more other polar organic solvents. In certain embodiments, the solvent may consist essentially of ethyl lactate, where no other components are present that materially affect the crystallization of PCL. In certain embodiments, the solvent may be substantially 100% ethyl lactate. It is further noted that upon precipitating PCL powder from a solution of PCL in ethyl lactate, a portion of the dissolved PCL may remain in solution. In certain embodiments, the addition of a secondary solvent which is miscible with the reprecipitation solvent but does not support dissolution of the PCL may be added to the PCL/solvent solution to induce precipitation. In certain embodiments, the use of a nucleating agent in powder form may be used to induce precipitation, and may help to control particle size and dispersity of particle size, and may help to improve the overall spheroidal shape of the powder particles. Separation of the precipitated PCL powder from the remainder of the solution therefore leaves a solution of ethyl lactate with a portion of dissolved PCL.

Ethyl lactate is a useful solvent for the process in that it dissolves PCL well; is shown herein to produce powder with characteristics well-suited to PBF 3D printing processes; has a boiling point well-separated from ambient temperature, allowing for a broad cooling range during precipitation; is miscible with commonly available and effective wash solvents (e.g., water or low molecular weight alcohols); has been shown to be relatively non-toxic in mammals (as exhibited in its use as a food additive); and may be broken down in the body to form ethanol and lactic acid.

In certain embodiments, the precipitated PCL powder has a D₈₅ particle size of less than 150 μm; specifically, a D₉₀ particle size of less than 150 μm. Certain embodiments include where the PCL powder has a D₉₀ particle size of less than 150 μm. A PCL powder in which 100% of the particles have a size of less than 150 μm may also be produced by this method. The PCL powder may also have a D₅₀ value of less than or equal to 100 μm. Specifically, the PCL powder may have a D₅₀ value of 10 μm to 100 μm. The average particle diameter of the PCL powder may also be less than or equal to 100 μm or include a D₅₀ value of between 0 to 100 μm.

In certain embodiments, a method of preparing an article comprises providing a powder composition comprising PCL powder, and using a powder bed fusing process with the powder composition to form a three-dimensional object. At least one PCL powder may have a D₅₀ particle size of less than 150 μm in diameter and is made by above-described methods. Embodiments include where the PCL powder has a D₉₀ particle size of less than 150 μm, a D₅₀ value of less than or equal to 100 μm, or a D₅₀ value of between 0 to 100 μm.

The PCL powder may be used as the sole component in the powder composition and applied directly in a powder bed fusing step. Alternatively, the PCL powder may first be mixed with other polymer powders, for example, another crystalline polymer or an amorphous polymer, or a combination of a semi-crystalline polymer and an amorphous polymer. The powder composition used in the powder bed fusing may include between 50 wt % to 100 wt % of the PCL powder, based on the total weight of all polymeric materials in the powder composition.

The PCL powder may also be combined with one or more additives/components to make a powder useful for powder bed fusing methods. Such optional components may be present in a sufficient amount to perform a particular function without adversely affecting the powder composition performance in powder bed fusing or the object prepared therefrom. Optional components may have a D₅₀ value which falls within the range of the average particle diameters of the PCL powder or an optional flow agent. If necessary, each optional component may be milled to a desired particle size and/or particle size distribution, which may be substantially similar to the PCL powder. Optional components may be particulate materials and include organic and inorganic materials such as fillers, flow agents, and coloring agents. Still other additional optional components may also include, for example, toners, extenders, fillers, colorants (e.g., pigments and dyes), lubricants, anticorrosion agents, thixotropic agents, dispersing agents, antioxidants, adhesion promoters, light stabilizers, organic solvents, surfactants, flame retardants, anti-static agents, plasticizers a combination comprising at least one of the foregoing. Yet another optional component also may be a second polymer that modifies the properties of the PCL powder. In certain embodiments, each optional component, if present at all, may be present in the powder composition in an amount of 0.01 wt % to 30 wt %, based on the total weight of the powder composition. The total amount of all optional components in the powder composition may range from 0 up to 30 wt % based on the total weight of the powder composition. Such an additive may also enhance the conversion of IR laser energy into thermal energy in the powder bed.

It is not necessary for each optional component to melt during the powder bed fusing process; e.g., a laser sintering process. However, each optional component may be selected to be homogeneously compatible with the PCL polymer in order to form a strong and durable object. The optional component, for example, may be a reinforcing agent that imparts additional strength to the formed object. Examples of the reinforcing agents include one or more types of glass fibers, carbon fibers, talc, clay, wollastonite, glass beads, and combinations thereof. Such an additive may also enhance the conversion of IR laser energy into thermal energy in the powder bed.

The powder composition may optionally contain a flow agent. In particular, the powder composition may include a particulate flow agent in an amount of 0.01 wt % to 5 wt %, specifically, 0.05 wt % to 1 wt %, based on the total weight of the powder composition. In certain embodiments, the powder composition comprises the particulate flow agent in an amount of 0.1 wt % to 0.25 wt %, based on the total weight of the powder composition. The flow agent included in the powder composition may be a particulate inorganic material having a median particle size of 10 μm or less, and may be chosen from a group consisting of hydrated silica, amorphous alumina, glassy silica, glassy phosphate, glassy borate, glassy oxide, titania, talc, mica, fumed silica, kaolin, attapulgite, calcium silicate, alumina, magnesium silicate, and combinations thereof. The flow agent may be present in an amount sufficient to allow the semi-crystalline polymer powder to flow and level on the build surface of the powder bed fusing apparatus (e.g., a laser sintering device). Such an additive may also enhance the conversion of IR laser energy into thermal energy in the powder bed.

The powder composition may optionally contain an IR-absorbing agent to facilitate the conversion of laser energy into thermal energy in the SLS process. The IR-absorbing agent may be one or more of a variety of inorganic or organic substances, such as metal oxides (e.g., titania, silica, glass, tungsten(VI) oxide), metal nanoparticles (e.g., gold nanorods), or organic compounds that absorb strongly at the wavelength of the IR laser (typically 10.6 μm, equivalent to 943 cm⁻¹).

Another optional component is a coloring agent, for example a pigment or a dye, like carbon black, to impart a desired color to the object. The coloring agent is not limited, as long as the coloring agent does not adversely affect the composition or an object prepared therefrom, and where the coloring agent is sufficiently stable to retain its color under conditions of the powder bed fusing process and exposure to heat and/or electromagnetic radiation; e.g., a laser used in a sintering process. Such an additive may also enhance the conversion of IR laser energy into thermal energy in the powder bed.

Still further additives include, for example, toners, extenders, fillers, lubricants, anticorrosion agents, thixotropic agents, dispersing agents, antioxidants, adhesion promoters, light stabilizers, organic solvents, surfactants, flame retardants, anti-static agents, plasticizers, and combinations of such. Such an additive may also enhance the conversion of IR laser energy into thermal energy in the powder bed.

Still another optional component also may be a second polymer that modifies the properties of the PCL powder.

The powder composition is a fusible powder composition and may be used in a powder bed fusing process such as selective laser sintering. An example of a selective laser sintering system for fabricating a part from a fusible powder composition, and in particular for fabricating the part from the fusible PCL powder disclosed herein, may be described as follows. One thin layer of powder composition comprising the PCL powder is spread over the sintering chamber. The laser beam traces the computer-controlled pattern, corresponding to the cross-section slice of the CAD model, to melt the powder selectively which has been preheated to slightly below its melting temperature. After one layer of powder is sintered, the powder bed piston is lowered with a predetermined increment (typically 100 μm), and another layer of powder is spread over the previous sintered layer by a roller. The process then repeats as the laser melts and fuses each successive layer to the previous layer until the entire object is completed. Three-dimensional objects comprising a plurality of fused layers may thus be made using the PCL powder described herein.

One or more variation may be constructed and arranged to provide one or more advantages, which may include, but not limited to, the use of a single solvent in preparing the PCL powder, which facilitates solvent recovery and reuse thereof. A number of variations, the PCL powder produced by at least one of the disclosed methods provides improved PBF performance. Additive manufacturing processes that employ fusion of a powder bed, including selective laser sintering (SLS), multi jet fusion (MJF), high speed sintering (HSS), and electrophotographic 3D-printing, may therefore benefit by forming and using PCL powder produced as described herein. In particular, the 3D printing of implantable, bioresorbable medical devices would benefit from the PCL powder material described herein.

In a number of variations, the reprecipitation process may serve to purify the PCL material, removing residual catalyst, initiator, monomer, and other contaminants. By dissolving the PCL, contaminants interstitially trapped in the solid are released into the resulting PCL solution. When the PCL reprecipitates, the quantity of contaminants that become reintercalated into the solid is significantly less, due both to a lower probability of entrapment, as well as the nature of the formation of crystallites to exclude contaminants. The reprecipitation process may be repeated with fresh, uncontaminated solvent to further reduce the level of contamination. A common contaminant to be removed from PCL is the tin compounds residual from the common use of a tin catalyst in the process of polymerizing c-caprolactone.

A number of variations may include a method of producing powder suitable for additive manufacturing, the method comprising: combining a polymeric material suitable and a solvent; dissolving the polymeric material suitable for additive manufacturing into the solvent to form a solution; cooling the solution to a temperature that causes at least a portion of the dissolved polymeric material suitable for additive manufacturing to precipitate from the solution; separating precipitated polymeric material from the solution; washing the separated, precipitated polymeric material to form a washed polymeric material; and drying the washed polymeric material to form a dry polymeric material suitable for additive manufacturing.

In at least one variation, polycaprolactone powder may be formed by dissolving polycaprolactone in a heated solvent. Alternatively, the solvent may not require heating. The solvent may be a non-toxic, biocompatible solvent. In at least one variation, the solvent may be ethyl lactate. A single solvent may be used. Reprecipitation solvents such as γ-valerolactone and ethyl acetate may be used. Reprecipitation systems such as xylene and petroleum ether, tetrahydrofuran and methanol, or dichloromethane and water may be used. Dispersants such as polyvinylpyrrolidone may also be employed in certain variations.

### EXAMPLES

Reprecipitation of Polycaprolactone Powder

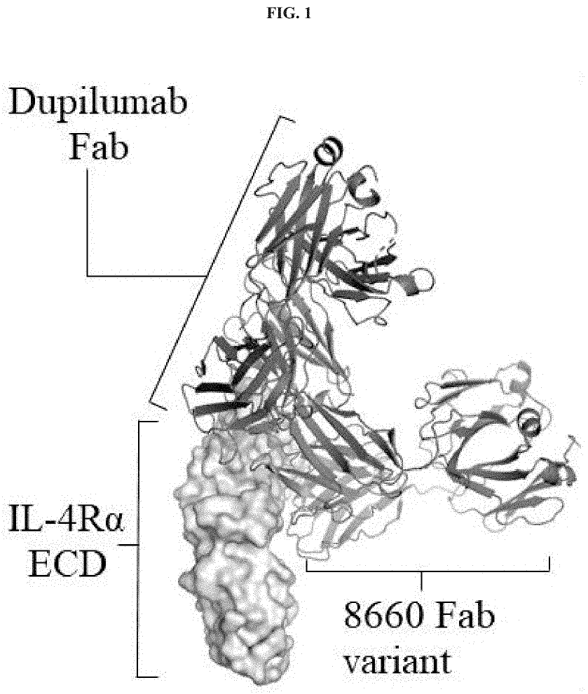

FIG. 1 illustrates a method of producing polycaprolactone powder, according to at least one variation. Polycaprolactone and solvent may be combined, for example as shown in step 101. Polycaprolactone pieces of any size may be used. Solvent may be one or more of the solvents described above. A single solvent may be used. Polycaprolactone may be heated before being added to the solvent to prevent the solvent temperature from decreasing upon polycaprolactone addition. The solvent may be heated. Optionally, the solvent may not require heating. In at least one variation, polycaprolactone may be heated above the polycaprolactone's melting point and then added to the solvent. The solvent may have a temperature that is also above the melting point of polycaprolactone. The polycaprolactone/solvent combination may be mixed, for example by stirring. A stir rate of 200 to 800 revolutions per minute may be used. In at least one variation, a stir rate of 600 to 700 rpm may be used. The concentration of polycaprolactone may range from 1% w/v to 20% w/v where the concentration of polycaprolactone is calculated by dividing the mass of polycaprolactone (in grams, g) by the volume of the solvent (in milliliters, ml). In some variations, polycaprolactone concentration may be (a) 13% w/v to 15% w/v or (b) 8% w/v to 10% w/v. Fresh or recycled (previously used for reprecipitating polycaprolactone) solvent may be used. At step 102, the temperature of the solvent/polycaprolactone combination may be controlled to a set point temperature. In one variation, the set point temperature may be between 60 and 145 degrees Celsius (including the end points of the range). In at least one variation, the set point temperature may range from 80 to 110 degrees Celsius (including the end points of the range). The set point temperature may be a temperature that is very close to (for example, within about five degrees Celsius) from the boiling point of the solvent. The boiling point of an ethyl lactate solvent may be about 154 degrees Celsius. The temperature of the solvent may be equal to the set point temperature. Polycaprolactone may dissolve into the solvent to create a solution as shown in step 102. Step 102 may proceed until all the polycaprolactone is dissolved. When all the polycaprolactone is dissolved, the solution may appear completely transparent and there may be minimal or no visible solids present.

At step 103, the temperature of the polycaprolactone solution may be reduced. A cooling step may lower the temperature through and below the saturation point of the solution which may cause dissolved polycaprolactone to precipitate out of solution. In one variation, the temperature of the polycaprolactone/solvent solution may be reduced to room temperature. At step 104, precipitated polycaprolactone may be separated from the solution. Separation may be accomplished by vacuum-filtration, for example, or by other separation techniques such as screening, centrifugation, cyclone separators, air classification, drying, etc. After the polycaprolactone is separated from the solution in step 104, the polycaprolactone may be washed in washing step 105. A miscible wash liquid such as water may be used to displace and/or extract residual solvent from the polycaprolactone. Wash liquid may be combined with polycaprolactone and the combination may be stirred. Alternatively, wash liquid may be sprayed over polycaprolactone solids that are positioned on top of a mesh or screen to displace and/or extract solvent and wash it from the polycaprolactone. Other liquid displacement or extraction methods may also be used in step 105. After polycaprolactone is washed, it may be dried. Polycaprolactone may be dried by heating to a temperature ranging from ambient (for example, 20 degrees Celsius) to 50 degrees Celsius. Polycaprolactone may be stationary as hot air (or other gas, such as nitrogen) passes over it to carry water vapor away. Alternatively, polycaprolactone may be tumbled or otherwise moved to improve mass transfer of wash liquid from the polycaprolactone to the surrounding environment during the drying step. A vacuum system may be used to decrease the pressure that the polycaprolactone is exposed to during the drying step to reduce the energy required for drying and/or to achieve more complete drying.

Dried polycaprolactone particles may be separated by size in step 107. Size separation may separate/isolate polycaprolactone particles that have a particle diameter within the range of 30 to 150 μm, 20 to 150 μm, or 1 to 150 μm. Size separation may separate polycaprolactone particles that have a particle diameter within a range that is desirable for a particular end use such as SLS printing. Size separation may be accomplished by screening, cyclone separation, air classifier, etc. Finally, the polycaprolactone or a sized fraction of the polycaprolactone may be used as a build material to manufacture an article. For example, a sized fraction of polycaprolactone may be used as a build material in an SLS printer to produce a 3D printed object. In at least one variation, the size separation step is excluded and the powder is used in an end-use application (for example, SLS printing) without performing a size separation step.

Powder Characterization

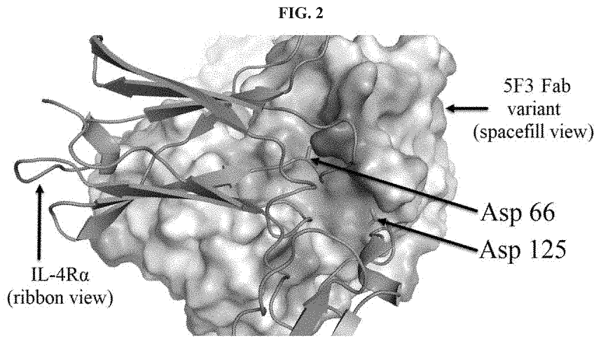

Polycaprolactone powder, precipitated according to the variations, resulted in the properties shown in FIGS. 2 through 5. FIG. 2 shows results from a thermal gravimetric analysis (TGA) that was performed on a sample of polycaprolactone produced according to at least one variation. The TGA analysis heats a sample and measures its weight as temperature is increased. The presence of residual solvent and thermal decomposition temperature may be ascertained by the TGA results because they might show up as a change in the rate of weight loss. The second plot (line) on the TGA graph is a derivative of the TGA curve and shows the rate of change in weight. As shown in FIG. 2, the onset of degradation in at least one variation began at 358° C. with 3% weight loss due to moisture.

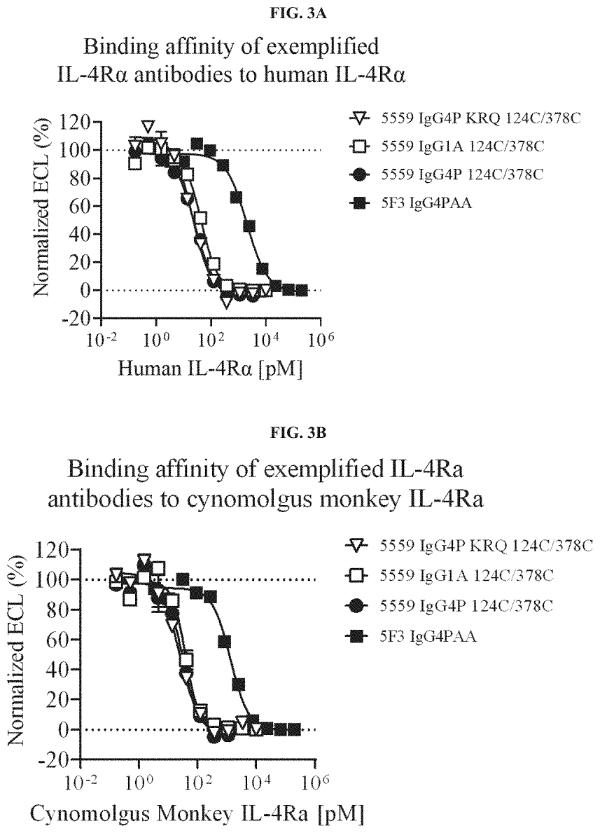

FIG. 3 shows a differential scanning calorimetry (DSC) curve of polycaprolactone precipitated according to at least one variation. As shown in the FIG. 3 variation, the onset of the first melt peak is at 49.81° C. with a peak temperature of 58.39° C. and an enthalpy of 101.86 J/g. The recrystallization peak in FIG. 3 has an onset at 25.87° C. with a peak at 21.02° C. and an enthalpy of 66.219 J/g. The second melt peak in FIG. 3 has an onset at 45.68° C. with an enthalpy of 55.090 J/g.

In a number of variations, using an ethyl lactate solvent may result in a polycaprolactone powder that has at least one of the following: (a) an onset of degradation temperature between about 287 and about 420 degrees Celsius, (b) a TGA mass loss between about 0 and about 3 mass %, (c) an onset of first melt between about 49 and about 58 degrees Celsius, (d) a first melt peak temperature between about 58 and about 65 degrees Celsius, (e) a first melt peak enthalpy between about 97 and about 111 J/g, (f) a recrystallization onset temperature between about 25 and about 34 degrees Celsius, (g) a recrystallization peak temperature between about 21 and about 28 degrees Celsius, (h) a recrystallization enthalpy between about 56 and about 67 J/g, (i) a second melt onset of melting temperature between about 45 and about 54 degrees Celsius, (j) a second melt peak temperature between about 51 and about 58 degrees Celsius, (k) a second melt enthalpy between about 26 and about 58 J/g, (l) a D₁₀ between about 32 and about 517 μm, when determined by volume percent, (m) a D₅₀ between about 50 and about 944 μm, when determined by volume percent (n) a D₉₀ between about 83 and about 1297 μm, when determined by volume percent (o) a volume percentage of particles having a diameter greater than 150 μm that is between about 0 and about 100 volume %, (p) a volume percentage of particles having a diameter that is less than 20 μm that is between 0 and 1 volume %, (q) a D₁₀ that is between about 14 and about 245 μm, when determined by number percent, (r) a D₅₀ between 24 and 359 μm, when determined by number percent, (s) a D₉₀ between 48 and 876 μm, when determined by number percent, (t) a number percentage of particles having a diameter greater than 150 μm that is between about 0 and about 100 number %, or (u) a number percentage of particles having a diameter less than 20 μm that is between about 0 and about 35 number %.

When interpreting the data, a Dₓ of y μm means that x percent of the particles in a sample had a particle size that was less than y μm. For example, a D₅₀ of 100 μm (when determined by volume percent) means that 50% (by volume) of the particles in a sample had a particle size that was less than 100 μm.

In variations using an ethyl lactate solvent, methods may produce a polycaprolactone powder that contains particles, wherein about 70 to about 100 volume percent of the particles have a particle diameter between 20 μm and 150 μm. Variations may produce a polycaprolactone powder that contains particles, wherein greater than 80 volume %, greater than 90 volume %, greater than 95 volume %, greater than 98 volume %, or even greater than 99 volume % of the particles have a particle diameter between 20 μm and 150 μm.

In variations using an ethyl lactate solvent, methods may produce a polycaprolactone powder that contains particles, wherein about 70 to about 100 number percent of the particles have a particle diameter between 20 μm and 150 μm. Variations may produce a polycaprolactone powder that contains particles, wherein greater than 80 number %, greater than 90 number %, greater than 95 number %, greater than 98 number %, or even greater than 99 number % of the particles have a particle diameter between 20 μm and 150 μm. When practicing the disclosed reprecipitation methods to produce a polycaprolactone powder, analytical methods such as NMR (nuclear magnetic resonance spectroscopy), GC (gas chromatograph), TGA (thermogravimetric analysis), etc. may be used to detect trace amounts of residual solvent in the polycaprolactone powder.

Using an ethyl acetate solvent may result in a polycaprolactone powder that has at least one of the following characteristics: (a) an onset of degradation temperature between about 329 and about 475 degrees Celsius, (b) a TGA mass loss between about 0 and about 0.5 mass %, (c) an onset of first melt between about 52 and about 57 degrees Celsius, (d) a first melt peak temperature between about 64 and about 67 degrees Celsius, (e) a first melt peak enthalpy between about 96 and about 105 J/g, (f) a recrystallization onset temperature between about 27 and about 31 degrees Celsius, (g) a recrystallization peak temperature between about 22 and about 26 degrees Celsius, (h) a recrystallization enthalpy between about 48 and about 63 J/g, (i) a second melt onset of melting temperature between about 50 and about 60 degrees Celsius, (j) a second melt peak temperature between about 56 and about 59 degrees Celsius, (k) a second melt enthalpy between about 50 and about 55 J/g, (l) a D₁₀ of about 28μm, when determined by volume percent, (m) a D₅₀ of about 1066 μm, when determined by volume percent (n) a D₉₀ of about 1283 μm, when determined by volume percent (o) a volume percentage of particles having a diameter greater than 150 μm that is about 67 volume %, (p) a volume percentage of particles having a diameter less than 20 μm that is about 1 volume %, (q) a D₁₀ that is about 46 μm, when determined by number percent, (r) a D₅₀ that is about 25 μm, when determined by number percent, (s) a D₉₀ that is about 3 μm, when determined by number percent, (t) a number percentage of particles having a diameter greater than 150 μm that is about 0 number %, or (u) a number percentage of particles having a diameter less than 20 μm that is about 33 number %.

Polycaprolactone produced according to at least one variation may have an intrinsic viscosity, as determined in chloroform at 25° C., of 0.3 to 3.0 deciliters per gram (dl/gm) (including the end points of this range). Polycaprolactone produced according to at least one variation may have an intrinsic viscosity, as determined in chloroform at 25° C., of 1.1 to 1.4 deciliters per gram (dl/gm) (including the end points of this range). The polycaprolactone may have a weight average molecular weight of 5,000 to 200,000 Daltons, specifically 100,000 to 150,000 Daltons, as measured by gel permeation chromatography (GPC), using a crosslinked styrene-divinylbenzene column and calibrated to polystyrene references. GPC samples are prepared at a concentration of 1 mg per mL (mg/mL), and are eluted at a flow rate of 1.5 mL per minute.

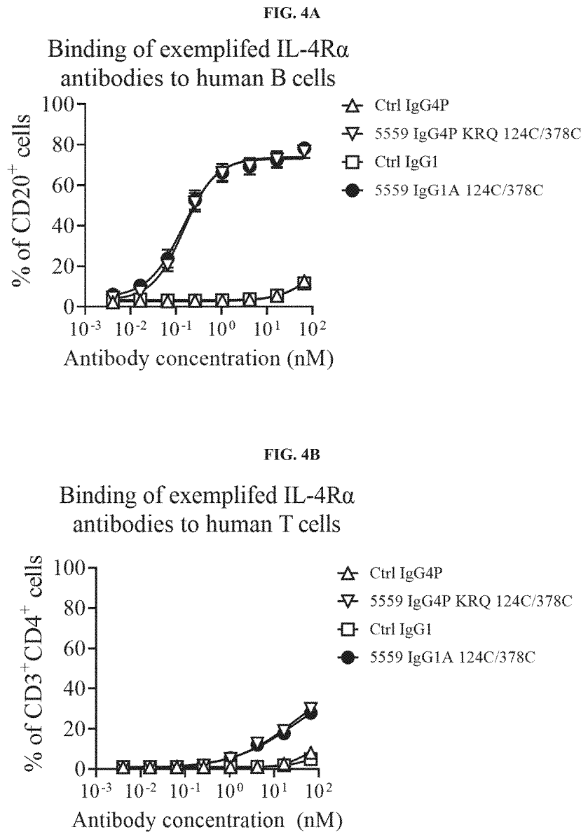

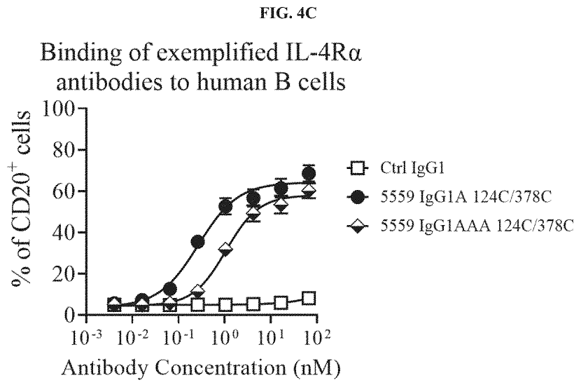

FIG. 4 shows the particle size volume distribution for an SLS-grade powder that was produced according to at least one variation. According to at least one variation, such as the variation shown in FIG. 4, the distribution may be almost Gaussian with a D₁₀ of 62.14 μm, a D₅₀ of 102.2 μm, and a D₉₀ of 156.6 μm.

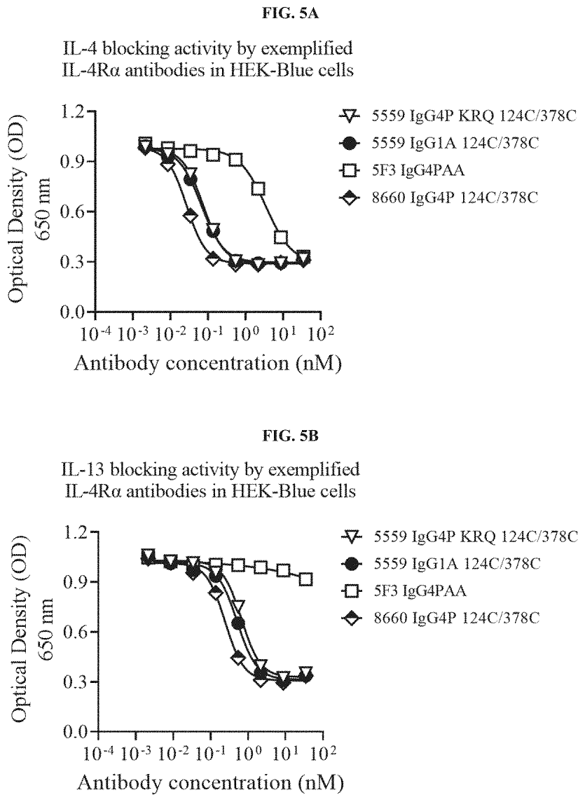

FIG. 5 shows the particle size number distribution for an SLS-grade powder that was produced according to at least one variation. The distribution may be relatively contained, with a drop off occurring around 100 μm; a D₁₀ of 31.23 μm, a D₅₀ of 59.88 μm, and a D₉₀ of 105.0 μm.

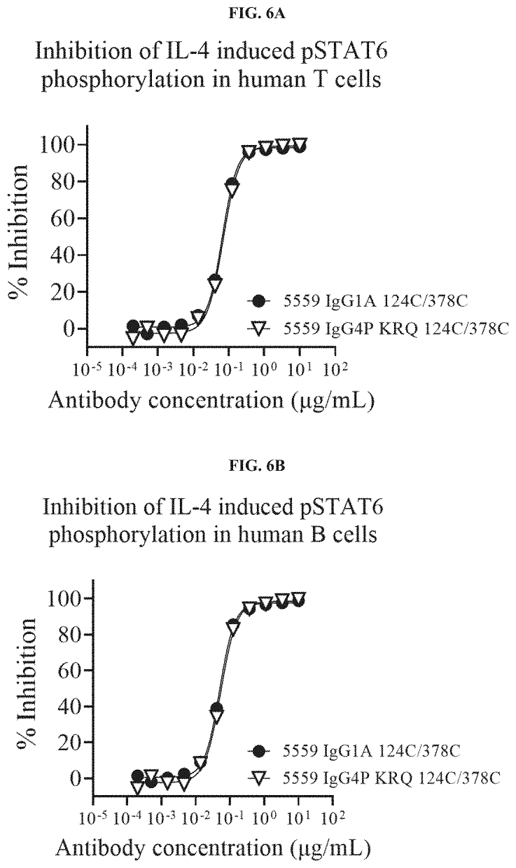

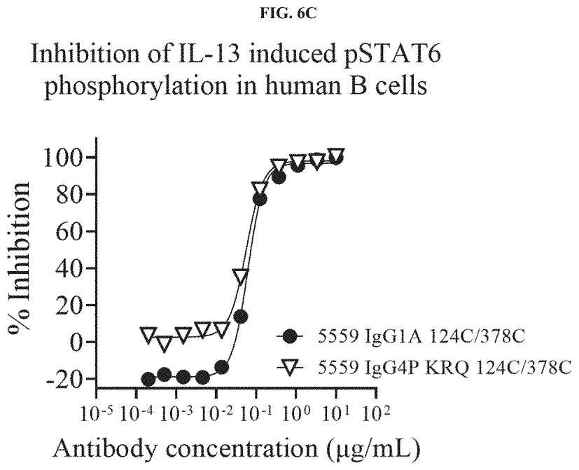

FIG. 6 shows powder data for a polycaprolactone powder that was produced according to at least one variation. FIG. 6 shows that the polycaprolactone powder, of at least one variation, had a melt peak temperature of 58.4° C. The figure also shows that the polycaprolactone, which was produced according to at least one variation, did not contain fine particles (defined as particles having a diameter less than 20 μm). This may be beneficial in some applications because fine particles may hinder the powder's ability to flow. The spheroidal character of the particles, produced according to at least one variation, was also tested. 90.54% v/v (volume/volume or “volume percent”) of the polycaprolactone particles had a sphericity greater than 0.75. 80.64% v/v of the polycaprolactone particles had a sphericity greater than 0.80. Sphericity values were calculated by equation 1, where Dₐ is defined in equation 2 and Dₚ is defined in equation 3. Higher sphericity values correlate with better ability to flow. In at least one variation, a polycaprolactone powder may be produced that has a Hausner ratio that is less than 1.25, where the Hausner ratio is defined as the ratio of tapped density to fluffy (bulk) density.

$$\frac{{D}_{a}}{{D}_{p}} = Sphericity \qquad ( Eq . 1 )$$

$${D}_{a} = Area equivalent diameter = {( \frac{4 * ( particle area )}{π} )}^{( \frac{1}{2} )} \qquad ( Eq . 2 )$$

$${D}_{p} = Perimeter equivalent diameter = \frac{Particle perimeter}{π} \qquad ( Eq . 3 )$$

SLS Testing

Polycaprolactone, produced according to at least one variation, may be blended with one or more other biocompatible components, such as hydroxyapatite. In at least one variation, hydroxyapatite may be added to polycaprolactone in an amount that is between 0.5% w/w and 10% w/w of the mass of the polycaprolactone. Hydroxyapatite is a mineral that is found in tooth enamel and bone and is used in bone tissue engineering. In variations, other components may be added to the polycaprolactone. Other components may include one or more types of glass fibers, carbon fibers, talc, clay, wollastonite, glass beads, or combinations thereof

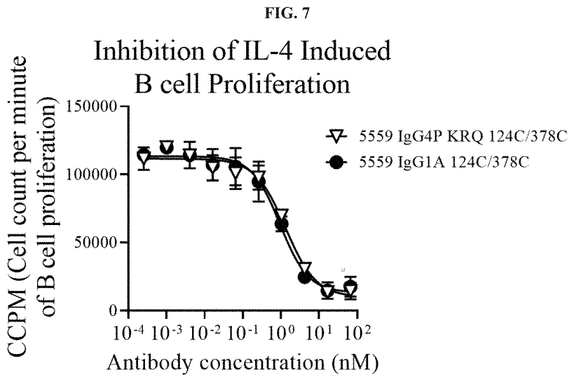

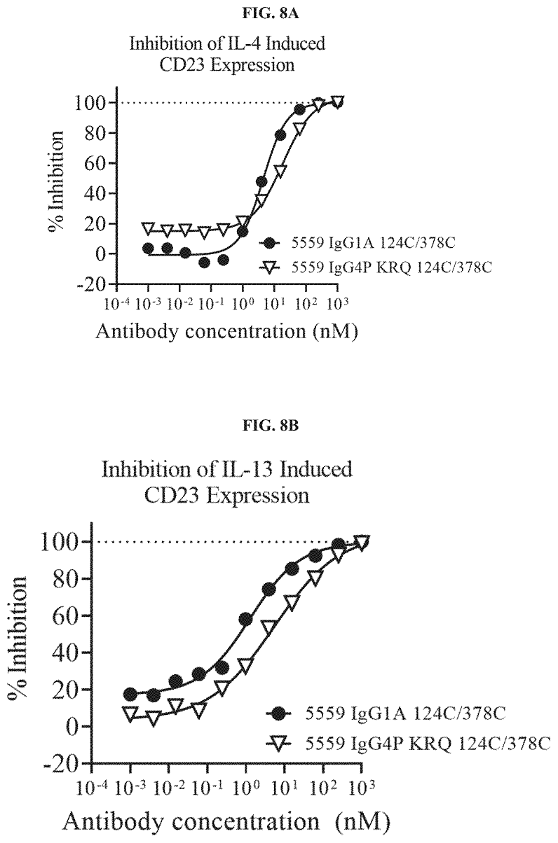

Polycaprolactone, produced according to at least one variation, was blended with 4% w/w hydroxyapatite (the mass of hydroxyapatite was 4% of the mass of polycaprolactone) and used in an SLS printer to produce tensile bars. Seven tensile bars were created with a part temperature of 56.5° C. and feed temperature of 40° C. using a double laser scan at 40 W with 0.18 mm scan spacing. The tensile bars were then pulled using the ASTM D_{638—}Type 4 tensile method. The pull rate was 5.00 mm/min. FIG. 7 shows a picture of the bars that were SLS printed using polycaprolactone that included 4% w/w hydroxyapatite (HA). FIG. 8a shows the tensile plot generated by pulling the SLS created polycaprolactone (with 4% w/w hydroxyapatite) tensile bars. FIG. 8B shows a summary of the material properties obtained from the tensile testing in FIG. 8A.

According to at least one variation, the moisture content of a polycaprolactone/hydroxyapatite powder may be adjusted prior to using the powder in an SLS machine. Water may aid the melting process by acting as a heat absorber. Hydroxyapatite may hinder the melting process by acting as a desiccant. Hydroxyapatite may facilitate the fusion of polycaprolactone powder due to its (hydroxyapatite's) IR-absorbing nature. Researchers have found that the amount of moisture (water) in a polycaprolactone/hydroxyapatite powder impacts the quality of SLS printed parts that are built with the material. Low polycaprolactone/hydroxyapatite moisture may be detrimental to part quality. To ensure good, printed part quality, water may be added to the polycaprolactone powders or polycaprolactone/hydroxyapatite blends of the variations. For example, the moisture content of the polycaprolactone powders or polycaprolactone/hydroxyapatite blends may be adjusted so that the powder(s) have an increased or decreased moisture content. Water content may be adjusted by adding water to the powder or by placing the powder in a humidity-controlled atmosphere, for example. Water content in a polycaprolactone powder or polycaprolactone/hydroxyapatite powder blend may be adjusted so that the moisture content of the powder is between 0.5 and 5% w/w. In at least one variation, a powder may contain about 3% w/w water (moisture).

Nucleators Such as Hydroxyapatite

In at least one variation, the solvent/polycaprolactone mixture may further include a nucleator. In at least one variation, the solvent/polycaprolactone mixture may further include hydroxyapatite as a nucleator.

### Example

Procedure:

A 250 mL Erlenmeyer flask was charged with ethyl lactate (100 mL) and heated to 80° C.

Once the set temperature was reached, polycaprolactone and hydroxyapatite were added with the polycaprolactone being at 12% w/v (g/mL) loading in the solvent and hydroxyapatite being 4% w/w of the polycaprolactone. The mixture was allowed to stir until the polymer was fully dissolved, then the heat was removed and reprecipitation was allowed to occur. Once the mixture had precipitated out such that the stir bar could not move, it was filtered to recover the solvent, washed in RT DI water for three hours, filtered, and allowed to air dry in an evaporation dish for 72 hours.

Observations:

Upon dissolution of the polycaprolactone, the solution remained opaque — likely due to the hydroxyapatite particles, which are not soluble in ethyl lactate.

The mixture precipitated within 2 hours of the heat being removed. On the 250 mL scale, this is twice as fast as a polycaprolactone solution without a nucleator. 43.5% of the ethyl lactate was recovered.

Powder Analysis:

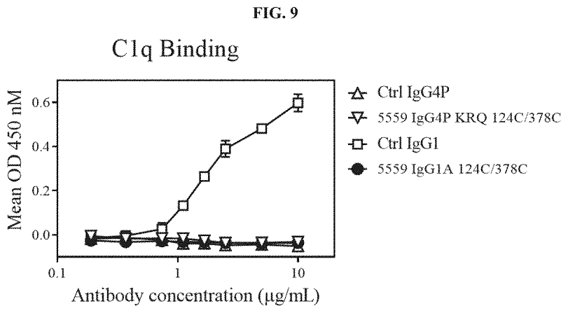

FIG. 9 shows a DSC curve for the resulting polycaprolactone powder nucleated by hydroxyapatite, according to at least one variation. The first melt curve has a peak at 62.42° C. and an enthalpy of 101.68 J/g. The recrystallization curve has a peak at 26.62° C. and an enthalpy of 59.38 J/g. The second melt curve has a peak at 57.80° C. and an enthalpy of 45.78 J/g.

FIG. 10 shows a particle number size distribution, according to at least one variation, with a D₁₀ of 19.14 μm, a D₅₀ of 30.58 μm, and a D₉₀ of 53.13 μm. Only 12.74% of all particles are outside the desired SLS range (where the desired SLS range is a particle diameter range between 20 μm and 150 μm), with 99.95% of all particles being smaller than 150 μm and 12.69% of particles being smaller than 20 μm.

FIG. 11 shows a particle number size distribution, according to at least one variation, with a D₁₀ of 31.47 μm, a D₅₀ of 61.25 μm, and a D₉₀ of 120.6 μm. Only 4.04% of all particles are outside the desired SLS range (the desired SLS range may be 20 μm-150 μm), with 96.87% of all particles being smaller than 150 μm and 0.91% of particles being smaller than 20 μm.

Comparison to Powder without a Nucleator:

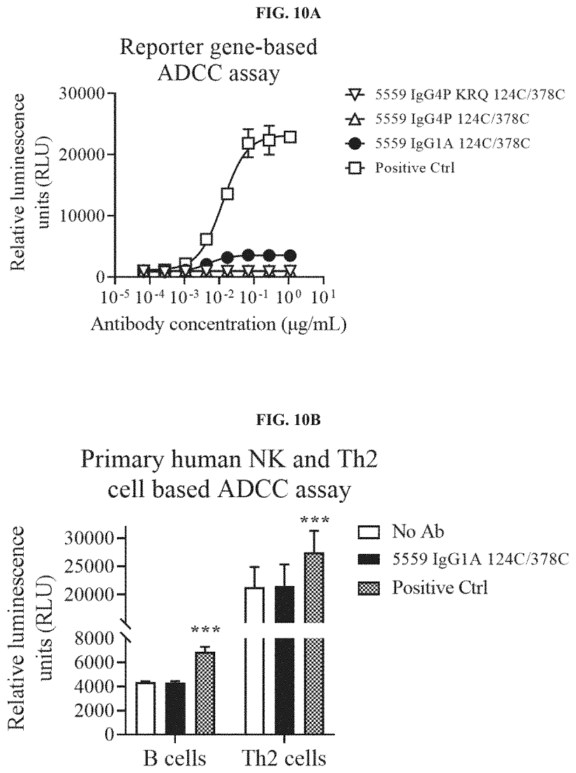

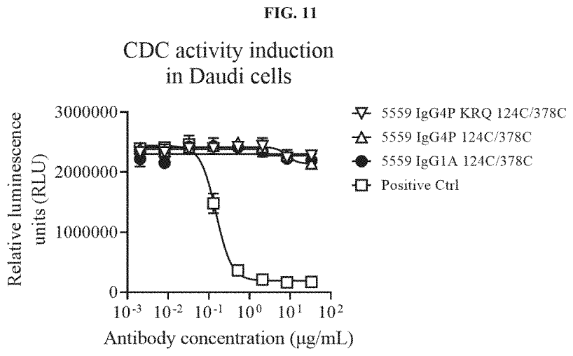

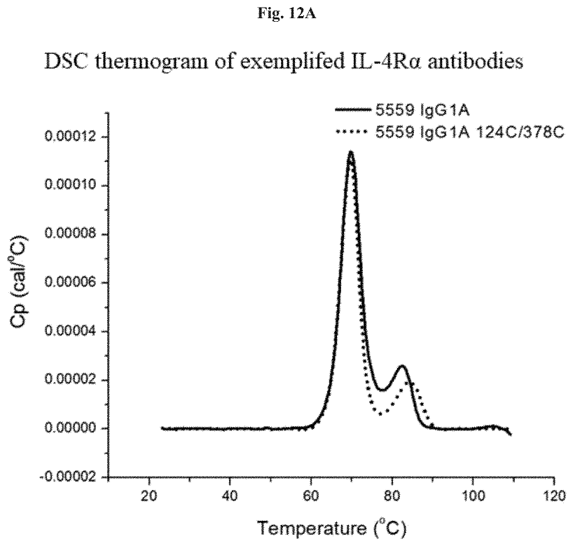

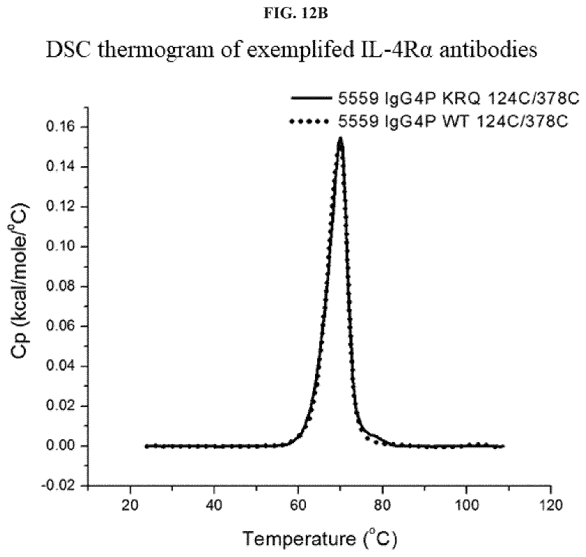

FIGS. 12A and 12B show a comparison between (12A) polycaprolactone powder nucleated with 4% w/w hydroxyapatite and (12B) polycaprolactone powder dry blended with 4% w/w hydroxyapatite and allowed to sit for over 24 hours. The figures (FIGS. 12A and 12B) compare pucks that were prepared by melting approximately 8 g of polycaprolactone/hydroxyapatite blend. Sample “(12A)” had 4% w/w hydroxyapatite added in before reprecipitation, allowing it to act as a nucleator. Sample “(12B)” had 4% w/w hydroxyapatite added after powder screening in a dry blend format.

In variations where hydroxyapatite is added as a nucleator, it may be added during a polycaprolactone precipitation step to form a solution comprising hydroxyapatite, polycaprolactone, and solvent. In at least one variation, an amount of hydroxyapatite may be added to the solvent/solution so that the hydroxyapatite is present in an amount that is between 0.5% w/w and 10% w/w of the mass of the polycaprolactone. Polycaprolactone may then precipitate from the solution to form a precipitated polycaprolactone powder that contains hydroxyapatite. This method may be used to prepare a puck such as the puck shown in FIG. 12A as puck “(12A).” The puck preparation method may comprise melting a polycaprolactone-containing material and then allowing the material to cool and solidify.

In variations where hydroxyapatite is dry blended with polycaprolactone, polycaprolactone may be precipitated from a solvent and dried. The dry polycaprolactone may then be blended with a certain amount of hydroxyapatite to form a powder comprising polycaprolactone and hydroxyapatite. This method may be used to prepare a puck such as the puck shown in FIG. 12B as puck “(12B).” The puck preparation method may comprise melting a polycaprolactone-containing material and then allowing the material to cool and solidify.

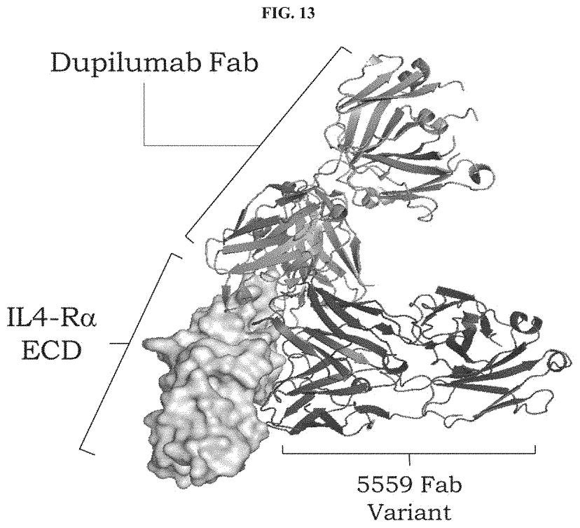

FIG. 13 shows a particle size distribution comparison between polycaprolactone precipitated on its own and polycaprolactone precipitated with hydroxyapatite acting as a nucleator. As shown in FIG. 13, adding hydroxyapatite as a nucleator in the reprecipitation step improves particle size distribution and may result in more polycaprolactone particles falling within the range of 20 to 150 μm.

When determining if a powder is suitable for the SLS, both the volume and number distributions are considered. In an ideal scenario, the volume and number distributions would be identical, with a D₅₀ of 60 μm and a distribution between 30 μm and 150 μm, with minimal powder outside of that range. However, in reality, this is unlikely to be the case. As such, the volume distribution is looked at to determine if the powder is suitable for SLS, and the number distribution looked at to determine if there are any potential issues that may arise. For example, too many particles smaller than 30 μm or smaller than 20 μm may cause flow issues whereas too many particles larger than 150 μm may cause resolution issues.

FIG. 13 shows a comparison between the particle size distributions of polycaprolactone precipitated with and without hydroxyapatite acting as a nucleator. Looking at the volume distributions, both powders have relatively Gaussian distributions, but the polycaprolactone precipitated with hydroxyapatite has an almost ideal D_{50.} Additionally, the polycaprolactone precipitated with hydroxyapatite has a smaller % of volume particles outside of the desired range (where a desired range may be a particle diameter size range between 20 μm and 150 μm diameter, including the end points of this range). Looking at the number distribution, the polycaprolactone precipitated with hydroxyapatite has 12.7% more particles smaller than 20 μm than the polycaprolactone without a nucleator. However, because of its near ideal volume distribution, the polycaprolactone powder with the nucleator may be preferred.

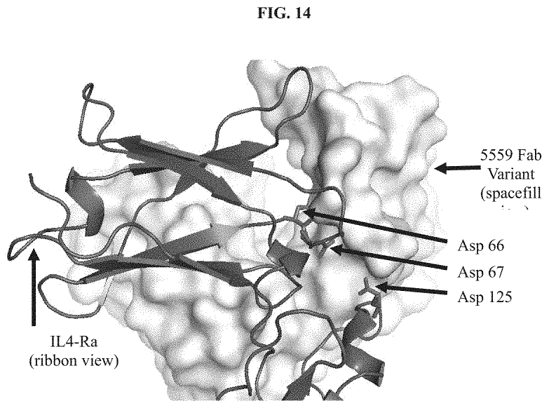

FIG. 14A shows a puck that was prepared by melting virgin polycaprolactone (at ambient moisture) in a convection oven. FIG. 14A shows two opposite sides (a top side and a bottom side) of a single puck, as do FIGS. 14B-G. FIG. 14B shows a puck that was prepared by melting virgin polycaprolactone (at ambient moisture) under IR (infrared). FIG. 14C shows a puck that was prepared by dry blending newly created polycaprolactone with 4% w/w hydroxyapatite and subsequently melting in a convection oven. FIG. 14D shows a puck that was prepared by dry blending newly created polycaprolactone with 4% w/w hydroxyapatite and subsequently melting under IR. FIG. 14E shows a puck that was prepared by dry blending 4% w/w hydroxyapatite with polycaprolactone, aging the blend at ambient conditions for 24 hours, and subsequently melting the aged blend in a convection oven. FIG. 14F shows a puck that was prepared by dry blending 4% w/w hydroxyapatite with polycaprolactone, aging the blend at ambient conditions for 24 hours, and subsequently melting the aged blend under IR. FIG. 14G shows a puck that was prepared by melting polycaprolactone in a convection oven, where the polycaprolactone was formed by a powder precipitation process that included 4% w/w hydroxyapatite as a nucleator (ie. hydroxyapatite was added to the solvent during precipitation to form a solution comprising hydroxyapatite, polycaprolactone, and solvent). As shown in 14G, adding hydroxyapatite as a nucleator during polycaprolactone precipitation in ethyl lactate produced a puck that was homogenous in appearance. In other words, without being bound by theory, using hydroxyapatite as a nucleator during the precipitation process appears to result in a melted object that may be well-mixed with a uniform concentration of hydroxyapatite and polycaprolactone throughout the melted object.

FIG. 15 shows a DSC curve for polycaprolactone powder that was reprecipitated in ethyl lactate. As shown in the figure, the polycaprolactone was first heated at a 20° C. per minute ramp rate to 100° C. The first melt peak temperature was 58.39° C., with an enthalpy of melting of 99.928 J/g. The polycaprolactone sample was then cooled at a cooling rate of 20° C. per minute to −10° C. The recrystallization peak temperature was 21.02° C., with an enthalpy of 62.261 J/g. The polycaprolactone sample was then heated a second time, at a rate of 20° C./min to 100° C. (as shown by the bottom dashed line in FIG. 15). The second heating cycle showed a melt peak temperature of 51.22° C., with an enthalpy of melting of 52.677 J/g.

FIG. 16 shows a DSC curve for polycaprolactone powder that was reprecipitated (in ethyl lactate) in the presence of 4% w/w hydroxyapatite as a nucleator, where the 4% w/w hydroxyapatite was calculated by diving the mass of hydroxyapatite that was added to the ethyl lactate by the mass of polycaprolactone that was added to the ethyl lactate. The DSC protocol used to generate the data in FIG. 16 was the same as the DSC protocol used to generate the data in FIG. 15—first, the sample was heated at a 20° C./min ramp rate to 100° C., then the sample was cooled at a rate of 20° C./min to −10° C., then the sample was again heated at a ramp rate of 20° C./min to 100° C. As shown in FIG. 16, the polycaprolactone sample that was reprecipitated in the presence of hydroxyapatite as a nucleator had a first melt peak temperature of 62.42° C., with an enthalpy of 101.68 J/g. The sample had a recrystallization peak temperature of 26.62° C., with an enthalpy of 59.376 J/g. Finally, the sample had a second peak melt temperature of 57.80° C., with an enthalpy of 45.775 J/g.

What is claimed is:

1. A powder comprising polycaprolactone particles, wherein greater than 90 volume percent of the polycaprolactone particles have a particle size that is between 20 microns and 150 microns, wherein the polycaprolactone particles contain a detectable amount of a nucleator, and wherein the polycaprolactone particles contain a detectable amount of a solvent comprising at least one of a biocompatible solvent or a bioresorbable solvent.

2. The powder of claim 1, wherein the solvent comprises ethyl lactate.

3. The powder of claim 1, wherein the nucleator is hydroxyapatite.

4. The powder of claim 1, wherein greater than 90 volume percent of the polycaprolactone particles have a sphericity that is greater than 0.75

5. The powder of claim 1, wherein greater than 80 volume percent of the polycaprolactone particles have a sphericity that is greater than 0.80.

6. The powder of claim 1, wherein the volume percent of polycaprolactone particles having a particle size less than 20 microns is zero or undetectable.

7. The powder of claim 1, wherein the powder has an enthalpy of melting of about 90 J/g to about 120 J/g.

8. A powder comprising polycaprolactone particles, a detectable amount of ethyl lactate, and a detectable amount of a nucleator; wherein the powder has a peak melting temperature of about 55° C. to about 65° C. and an enthalpy of melting of about 90 J/g to about 120 J/g.

9. The powder of claim 8, wherein the nucleator is hydroxyappetite.

10. The powder of claim 8, wherein the powder has a recrystallization peak of about 15° C. to about 35° C.

11. The powder of claim 8, wherein the powder has an onset of degradation temperature of about 250° C. to about 425° C.

12. The powder of claim 8, wherein greater than 96 number percent of the polycaprolactone particles have a particle size that is less than 125 microns.

13. The powder of claim 8, wherein greater than 90 volume percent of the polycaprolactone particles have a sphericity that is greater than 0.75.

14. A powder comprising polycaprolactone particles having a detectable amount of ethyl lactate and a detectable amount of a nucleator, wherein greater than 96 number percent of the polycaprolactone particles have a particle size that is less than 125 microns and wherein greater than 90 volume percent of the polycaprolactone particles have a sphericity that is greater than 0.75 and wherein the polycaprolactone particles have a moisture content that is adjusted to and maintained between 0.5% w/w and 5 w/w.

15. The powder of claim 14, wherein the nucleator is hydroxyapatite.

16. A method of producing polycaprolactone powder, the method comprising:
combining polycaprolactone and a polar organic solvent;
dissolving the polycaprolactone into the polar organic solvent to form a solution;
cooling the solution to a temperature that causes at least a portion of the dissolved polycaprolactone to precipitate from the solution;
adding a nucleator to the solution;
separating precipitated polycaprolactone from the solution;
washing the separated, precipitated polycaprolactone to form a washed polycaprolactone; and
drying the washed polycaprolactone to form a dry polycaprolactone.

17. The method of claim 16, further comprising heating the combined polycaprolactone and the polar organic solvent.

18. The method of claim 16, further comprising a separation step that separates dry polycaprolactone particles having a particle size less than 150 microns from larger dry polycaprolactone particles to form a sized polycaprolactone.

19. The method of claim 18, wherein the percent of nucleator in the combined polycaprolactone/nucleator mixture is between about 0.5 mass percent and 10 mass percent.

20. The method of claim 16, wherein the nucleator is hydroxyappetite.

21. The method of claim 16, wherein greater than 90 volume percent of the sized polycaprolactone particles have a sphericity that is greater than 0.75.

22. The method of claim 16, wherein greater than 80 volume percent of the sized polycaprolactone particles have a sphericity that is greater than 0.80.

23. The method of claim 16, wherein the polar organic solvent is selected from the group consisting of: ethyl acetate, ethyl lactate, γ-valerolactone, N,N-dimethylformamide (DMF), N-methyl-2-pyrrolidone (NMP), tetrahydrofuran (THF), dichloromethane (DCM), chloroform;
acetone, and dimethyl sulfoxide (DMSO).

24. The method of claim 23, wherein the polar organic solvent is ethyl lactate.

25. A method of producing polycaprolactone powder, the method comprising:
combining polycaprolactone and ethyl lactate;
dissolving the polycaprolactone and at least one nucleator into the ethyl lactate to form a solution;
cooling the solution to a temperature that causes at least a portion of the dissolved polycaprolactone to precipitate from the solution;
separating precipitated polycaprolactone from the solution;
washing the separated, precipitated polycaprolactone to form a washed polycaprolactone; and
drying the washed polycaprolactone to form a dry polycaprolactone.

26. The method of claim 25, wherein the at least one nucleator comprises hydroxyapatite.

27. The method of claim 25, wherein the solution is heated.

28. A method of additive manufacturing, the method comprising:
selectively melting or sintering adjacent polycaprolactone particles,
wherein greater than 96 number percent of the polycaprolactone particles have a particle size that is less than 125 microns and wherein greater than 90 volume percent of the polycaprolactone particles have a sphericity that is greater than 0.75,
wherein the polycaprolactone particles contain a detectable amount of hydroxyappetite, and
wherein the polycaprolactone particles contain a detectable amount of ethyl lactate.

29. The method of claim 28, wherein the polycaprolactone particles have a moisture content that is adjusted to and maintained between 0.5 and 5% w/w.

30. An article comprising polycaprolactone particles, wherein greater than 90 volume percent of the polycaprolactone particles have a particle size that is between 20 microns and 150 microns, wherein the polycaprolactone particles contain a detectable amount of a nucleator, and wherein the polycaprolactone particles contain a detectable amount of a solvent comprising at least one of a biocompatible solvent or a bioresorbable solvent.

31. A medical product comprising polycaprolactone particles, wherein greater than 90 volume percent of the polycaprolactone particles have a particle size that is between 20 microns and 150 microns, wherein the polycaprolactone particles contain a detectable amount of a nucleator, and wherein the polycaprolactone particles contain a detectable amount of a solvent comprising at least one of a biocompatible solvent or a bioresorbable solvent.
